(19) United States
(12) Patent Application Publication
(10) Pub. No.: US 2011/0009464 A1
(43) Pub. Date: Jan. 13, 2011

(54) IMMUNE SYSTEM STIMULATION BY LIGHT THERAPY INDUCED APOPTOTIC CELL DEATH IN ABNORMAL TISSUE

(75) Inventor: James C. Chen, Clyde Hill, WA (US)

Correspondence Address: LAW OFFICES OF RONALD M ANDERSON, 600 108TH AVE, NE, SUITE 507, BELLEVUE, WA, 98004, US

(73) Assignee: Light Sciences Oncology, Inc., Bellevue, WA (US)

(21) Appl. No.: 12/817,068

(22) Filed: Jun. 16, 2010

Related U.S. Application Data
(60) Provisional application No. 61/224,186, filed on Jul. 9, 2009.

(51) Int. Cl. A61K 31/407; A61P 35/00

(52) U.S. Cl. 514/410

(57) ABSTRACT

The efficacy of light activated therapy treatment is enhanced by stimulating the immune system of the patient substantially above the pre-therapy level. Abnormal tissue that is destroyed by the light activated therapy releases factors that stimulate the immune system, leading to systemic reductions in abnormal tissue (i.e., reduction beyond the area treated using light), so long as the light therapy conditions favor apoptosis over necrosis. The volume of abnormal tissue destroyed is maximized to the extent possible, reducing tumor load, which reduces an amount of immunosuppressive factors in the body, enabling stimulation of the immune system to be successful.

### RELATED APPLICATIONS

This application is based on a prior copending provisional application Ser. No. 61/224,186, filed on Jul. 9, 2009, the benefit of the filing date of which is hereby claimed under 35 U.S.C. §119(e).

### BACKGROUND

Abnormal cells in the body are known to selectively absorb certain dyes that have been perfused into a treatment site to a much greater extent than absorbed by surrounding tissue. For example, tumors of the pancreas and colon may absorb two to three times the volume of certain dyes, compared to normal cells. Once pre-sensitized by dye tagging in this manner, the cancerous or abnormal cells can be destroyed by irradiation with light of an appropriate wavelength or waveband corresponding to an absorption wavelength or waveband of the dye, with minimal damage to surrounding normal tissue. The procedure that uses light to destroy undesirable tissue, known as light activated drug therapy, has been clinically used to treat metastatic breast cancer, bladder cancer, lung carcinomas, esophageal cancer, basal cell carcinoma, malignant melanoma, ocular tumors, head and neck cancers, and other types of malignant tissue growths. Because this therapy selectively destroys abnormal cells that have absorbed more of a photoreactive dye than normal cells, it can successfully be used to kill malignant tissue with less effect on surrounding benign tissue than alternative treatment procedures.

In typical applications, the light is administered to an internal treatment site through an optical fiber from an external source such as a laser, or is applied to a site exposed during a surgical procedure. However, alternative techniques exist to provide light therapy. For example, several different embodiments of implantable light emitting probes for administering light activated therapy to an internal site within a patient's body are disclosed in commonly assigned U.S. Pat. No. 5,445,608. Further, a number of embodiments of flexible light emitting probes are disclosed in commonly assigned U.S. Pat. Nos. 5,800,478, 5,766,234, and 5,876,427. The above-referenced U.S. Pat. No. 5,445,608 teaches that an implantable probe containing a plurality of light sources can be transcutaneously introduced to a desired treatment site through a surgical incision and then left in place for an extended period of time so that the light emitted by light emitting diodes (LEDs) or other types of light sources mounted in the probe can administer light activated therapy to destroy abnormal tissue or other types of pathogenic organisms that have absorbed an appropriate photoreactive agent. Similarly, the flexible microcircuits disclosed in the above-noted patents are generally intended to be introduced into the body through a natural opening or through a small incision and positioned at the treatment site using conventional endoscopic techniques. The flexibility of these microcircuits facilitates their insertion into the body and disposition at the treatment site. Additional light emitting probes are disclosed in commonly assigned U.S. Pat. No. 6,416,531, U.S. patent application Ser. No. 11/416,783, and U.S. patent application Ser. No. 12/445,061.

It has been recognized that damaged or dead tumor cells can release various immune stimulating factors. This fact would suggest that killing tumor cells would result in a stimulated immune response that would lead to a further reduction in tumor cells, a desirable outcome.

In practice however, the tumor microenvironment has proven to be very immuno-suppressive, to the extent that the desired and expected immune-stimulating effect is rarely achieved. It would be desirable to provide methods for killing tumor cells that more readily achieve an overall immune stimulating effect.

### SUMMARY

In accord with the concepts disclosed herein, a method is defined for more effectively destroying abnormal tissue in a mass of abnormal tissue at a treatment site within a patient's body, to stimulate the body's immune system to lead to greater reductions in the amount of abnormal tissue present in the patient's body.

The techniques disclosed herein focus on controlling a relative type of abnormal cell death induced by light therapy, a relative amount of abnormal tissue initially killed by the light therapy, and a relative location of the abnormal tissue that is initially killed by the light therapy, such that the overall tumor microenvironment shifts from being immunosuppressive to immunogenic.

These techniques were developed based on recognizing that the tumor microenvironment is naturally strongly immunosuppressive, and naturally resistant to change. For example, necrotic cell death in abnormal tissue releases various tumor growth factors, which lead to rapid tumor re-growth. While the necrotic cells likely release immune-stimulating factors, the rapid tumor re-growth quickly reestablishes an immunosuppressive microenvironment, and little or no overall immune-stimulating effect is achieved. Thus, the concepts disclosed herein emphasize light therapy conducted to maximize apoptotic death of abnormal cells, while minimizing necrotic cell death.

Furthermore, applicants have recognized that if only a relatively small amount of abnormal tumor cells are killed at any one time, while such dead cells will release immune-stimulating factors, the amount of immune-stimulating factors released will be insufficient to overcome the immunosuppressive microenvironment established by the surviving tumor cells. Even where a relatively larger amount of abnormal cells are killed by a therapeutic treatment, unless those dead tumor cells are concentrated in a relatively small area, the naturally immunosuppressive tumor microenvironment is likely to remain intact. This is analogous to the concept that a relatively large force spread over a relatively larger area may be insufficient to penetrate a barrier, whereas the same force focused on a much smaller area will lead to such penetration. Finally, applicants have recognized that the location of the dead cells must be selected such that at least some of the dead cells are proximate a boundary of a tumor mass, such that the immune stimulating factors can disperse into the body and reach the patient's lymphatic system.

Thus, the concepts herein are directed to conducting light therapy on a mass of abnormal tissue to induce apoptotic cell death in a contiguous portion of the mass while minimizing necrotic cell death, where at least some of the apoptotic cells are proximate an outer boundary of the mass. Achieving the goal of maximizing apoptosis while minimizing necrosis will generate immune-stimulating factors, while minimizing the production of tumor growth factors (which would result in increased tumor growth and a more immunosuppressive microenvironment). Concentrating the mass of apoptotic cells in a contiguous portion will facilitate concentrating the immune-stimulating factors to overcome the immunosuppressive microenvironment in one area of the tumor mass. Ensuring that the contiguous portion is proximate to the boundary of the tumor will ensure that the immune-stimulating factors will reach the patient's lymphatic systems.

In at least one exemplary embodiment, the contiguous portion comprises about 50% of the tumor. The purpose of this is to substantially reduce the amount of viable tumor cells (tumor load) that are secreting immunosuppressive factors. Unless the tumor load is reduced, the balance between the immune-stimulating factors produced by the dead tumor cells and the immunosuppressive factors released by the live tumor cells is unlikely to result in an overall immune-stimulating environment. In other words, without substantially reducing the tumor load, the generation of immune-stimulating factors is unlikely to result in an overall immune-stimulating effect. In general, the contiguous volume should be as large as practical. To enable relatively larger contiguous volumes to be treated, a plurality of light probes can be implanted into the tumor, such that the fluence zone of each light probe overlaps the fluence zone of at least one other light probe.

Significantly, the establishment of an overall immune-stimulating environment that counteracts and exceeds the immunosuppressive environment of the tumor will lead to the death of additional tumor cells (i.e., the death of tumor cells beyond those deaths caused by the light therapy) at the tumor treated with the light therapy, as well as the death of abnormal cells elsewhere in the patient's body, due to the action of the patient's stimulated immune system.

Another aspect of the concepts disclosed herein is resetting the immune system to an earlier, more functional state. As tumor mass and volume increases, the immunosuppressive nature of tumor cells begins to overload the body's immune system, such that the immune system is no longer capable of attacking the tumor cells. The techniques disclosed herein (inducing apoptotic tumor cell death in a contiguous and substantial portion of a tumor mass, to simultaneously reduce tumor load and stimulate the immune system) can be considered to reset the immune system to a more functional state, such that the body's natural defenses are reinvigorated and are able to go on the offensive against the tumor cells, both in the initially treated tumor mass and elsewhere in the body.

More specifically, the present approach is directed to a method for destroying abnormal tissue in an abnormal tissue mass in a patient. The method includes the step of administering a photoreactive agent having one or more characteristic light absorption wavebands, to the patient such that a quantity of the photoreactive agent is present in the abnormal tissue mass. A contiguous portion of the abnormal tissue mass is then irradiated with light having a characteristic wavelength or waveband that overlaps at least a portion of at least one characteristic absorption waveband of the photoreactive agent. At least a portion of the contiguous portion of the abnormal tissue mass is disposed proximate to an outer boundary of the abnormal tissue mass. Conditions for irradiating the contiguous portion of the abnormal tissue mass with the light are controlled so as to reduce a release of immunosuppressive factors by the abnormal tissue, while stimulating a release of immune-stimulating factors by apoptotic cell death in the abnormal tissue mass.

The step of controlling the conditions for irradiating can comprise the step of controlling light fluence while irradiating the contiguous portions of the abnormal tissue mass so that the fluence is at a level selected to preferentially cause apoptotic cell death rather than necrotic cell death in the abnormal tissue mass. The step of controlling the conditions for irradiating can include the step of reducing a number of viable cells in the abnormal tissue mass while causing minimal necrotic cell death of the abnormal tissue.

The method further includes the step of stimulating an immunogenic response by immune system of the patient, with the release of the immune-stimulating factors by the apoptotic cells in the abnormal tissue mass. This step enables the immune system to attack remaining abnormal tissue both in the abnormal tissue mass and elsewhere in the patient.

The step of stimulating the immunogenic response can include the step of achieving at least one clinical endpoint selected from a group of clinical endpoints. The group consists of increasing an overall survival rate of the patient, increasing a medial overall survival rate of the patient, increasing a progression free survival rate of the patient, increasing a disease free survival rate of the patient, generating a positive post treatment tumor response in the patient, providing relief of symptoms associated with the abnormal tissue mass, reducing symptoms in the patient that are associated with the abnormal tissue mass, providing a clinical benefit to the patient, and reducing a degree of cachexia in the patient.

The method can also include the step of introducing a plurality of light probes into the abnormal tissue mass to emit light used for irradiating the contiguous portion of the abnormal tissue mass. The plurality of light probes can be positioned so that they are generally adjacent to each other. Also, the method can include the step of overlapping fluence zones of at least some of the plurality of light probes, enabling irradiation of the contiguous portion of the abnormal tissue mass with the light emitted by the plurality of light probes.

The step of irradiating the contiguous portion of the abnormal tissue mass with light can comprise the step of irradiating a continuous portion of the abnormal tissue mass that corresponds to about 50% to about 99% of the abnormal tissue mass, or more preferably, about 75% to about 99% of the abnormal tissue mass, but at least 20% of the abnormal tissue mass.

This application hereby specifically incorporates herein by reference the disclosures and drawings of each patent application and any issued patent identified above as a related application.

This Summary has been provided to introduce a few concepts in a simplified form that are further described in detail below in the Description. However, this Summary is not intended to identify key or essential features of the claimed subject matter, nor is it intended to be used as an aid in determining the scope of the claimed subject matter.

### DRAWINGS

Various aspects and attendant advantages of one or more exemplary embodiments and modifications thereto will become more readily appreciated as the same becomes better understood by reference to the following detailed description, when taken in conjunction with the accompanying drawings, wherein:

FIG. 1 is schematic illustration of a tumor, showing an implanted light source delivering a light activated therapy treatment internally to the tumor, where a light activated agent has been administered in association (i.e., either before, concurrently with, or a combination therefore) with light activated therapy;

FIG. 2 is a schematic drawing illustrating a side elevational view of light therapy using a plurality of light probes to deliver light activated therapy treatment to a contiguous portion of a tumor;

FIG. 3 is a plan view of the tumor shown in FIG. 1, illustrating the positions of probes and the radial depth to which light emitted thereby directly penetrates into the tumor;

FIG. 4 is a plan view of the tumor shown in FIGS. 1, 2, and 3, illustrating the direct light penetration pattern for a different configuration of a plurality of light probes;

FIG. 5 schematically illustrates an exemplary light emitting probe having a plurality of light sources contained therein; and

FIG. 6 graphically illustrates an exemplary timeline for the treatment disclosed herein.

### DESCRIPTION

### Figures and Disclosed Embodiments Are Not Limiting

Exemplary embodiments are illustrated in referenced Figures of the drawings. It is intended that the embodiments and Figures disclosed herein are to be considered illustrative rather than restrictive. No limitation on the scope of the technology and of the claims that follow is to be imputed to the examples shown in the drawings and discussed herein. Further, it should be understood that any feature of one embodiment disclosed herein can be combined with one or more features of any other embodiment that is disclosed, unless otherwise indicated.

Tumor cells can be killed in two general manners, apoptosis and necrosis. Apoptosis is a programmed, gradual cell death, which occurs when a cell is old or damaged. Necrosis is a sudden cell death, which can be caused by many different treatment strategies, such as trauma to remaining tissue after the surgical removal of unwanted tissue, the cooling of tissue via cryoablation, or the heating of tissue via microwaves (or some other energy source), which kill cells rapidly.

Necrotic cell death results in the release of inflammatory agents, whereas no such inflammatory agents are released in apoptosis. The inflammatory agents trigger a natural physiological response intended to stimulate tissue re-growth, to enhance wound healing. Unfortunately, this stereotypic wound healing process can influence tumor cells locally and remotely, and in a tumor, this response may stimulate tumor re-growth. Thus, the techniques disclosed herein maximize apoptotic cell death and minimize necrotic cell death.

When tumor cells are killed, they also release immunogenic agents that can stimulate the immune system. However, little if any clinical benefit typically accrues from this response in most patients with significant tumor load, because the immunosuppressive properties of the tumor overwhelm the immunogenic effect. Thus, the techniques disclosed herein emphasize reducing the tumor load by inducing tumor cell death in a contiguous portion of a tumor, while maximizing the immunogenic response by ensuring that at least some of the contiguous portion is proximate a boundary of the tumor, so the immune-stimulating factors released during tumor cell death can reach the patient's lymphatic system.

In an exemplary embodiment, light is used to activate a drug (talaporfin sodium) present in the tumor, which damages tumor cells and induces vascular infarction, resulting in tumor apoptosis. Such programmed cell death generates both cytolytic and memory CD8+T cells demonstrated in the preclinical setting. These immune effector cells are tumor specific and active against metastatic tumor cells. The systemic magnitude of the autologous vaccine-like action will depend on the number of effector T cells generated versus the volume of the tumor. The greater the tumor volume killed by the therapy, the greater the amount and concentration of breakdown products available for processing by the lymph nodes, spleen, and other lymphoid tissues. Those of ordinary skill in the art will recognize that the photoreactive drug can be administered in a variety of ways, such as direct introduction into the tumor, or systematic introduction into the body, which with time will result in accumulation of the drug in the tumor. The concepts disclosed herein are not limited to a particular method of drug delivery. It should be noted that in some embodiments, the photoreactive drug will be absorbed by the tissue in the tumor mass. However, it should be recognized that where the photoreactive drug will damage blood vessels when activated, absorption into tumor tissue is not required, so long as the photoreactive drug is present in blood vessels in the tumor, and the activating light reaches the photoreactive drug in those blood vessels. The claims that follow refer to a photoreactive drug that is present in the abnormal tissue mass. That phrase is intended to encompass both a photoreactive agent/drug that is absorbed by the abnormal tissue, and a photoreactive agent/drug that is present in blood vessels in the abnormal tissue mass.

To maximize the tumor volume killed by the therapy, in at least one embodiment, a plurality of light sources is used, to enable a relatively larger contiguous volume of the tumor to be irradiated with light to activate the drug in the tumor. Note that spacing individual light devices too widely “dilutes” the total light dose, undesirably reducing the total amount of tumor undergoing apoptosis.

The size of the contiguous volume will vary due to the location and size of the tumor. In general, it is preferable to treat as much tumor volume as possible to maximize numbers of T cells generated, to reduce the tumor load, and to reduce the number of tumor cells needed to be killed immunologically. Maximal reduction of a viable tumor directly (activation of the drug) and indirectly (immune effect) improves the ability of this technique to enable the patient's body to mount an effective immune response.

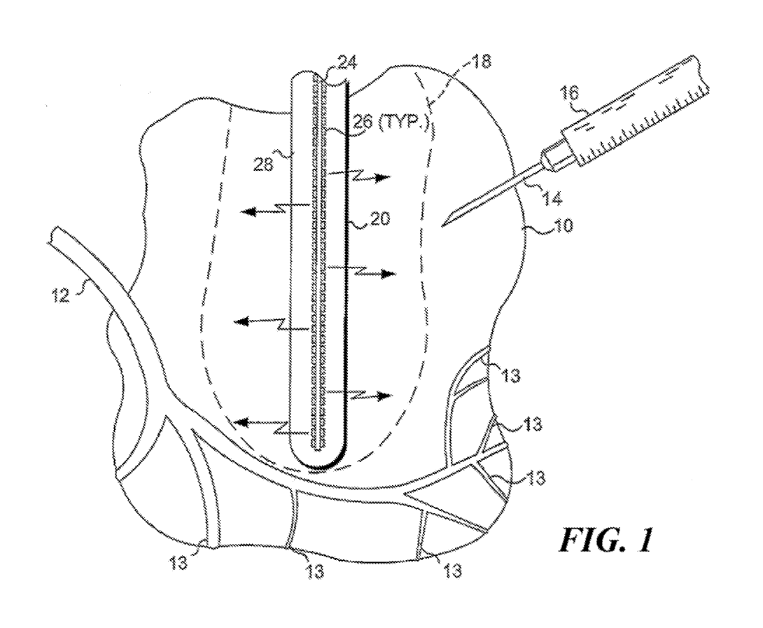

FIG. 1 schematically illustrates how the concepts disclosed herein are employed to achieve light activated therapy treatment of a tumor 10. In FIG. 1, tumor 10 is supplied with blood through one or more main vessels 12, having a plurality of branching vessels 13. Only one such main vessel is illustrated to simplify the Figure. Because the cells comprising tumor 10 are abnormal, it tends to grow at a relatively rapid rate and if left unchecked, the condition may lead to a metastatic spread of the abnormal cells throughout a patient's body.

To administer light activated therapy treatments to tumor 10 in the example shown in FIG. 1, an elongate probe 20 is implanted internally within tumor 10 during a conventional minimally invasive, surgical, or endoscopic procedure. Probe 20 may be either rigid or flexible, as appropriate to the technique used to facilitate its placement within tumor 10 and depending upon the location of the tumor within the patient's body. Probe 20 includes a plurality of light sources 26, e.g., LEDs, which are disposed on opposite sides of a substrate 24 (or disposed on a single side of a transparent substrate). It should be noted that the concepts disclosed herein are not limited to a particular method of light delivery. Details such as the electrically conductive traces that convey electrical current to each of the light sources are not shown. An optically transparent and biocompatible sheath 28 encloses light sources 26 and substrate 24, but allows light emitted by the light sources to be transmitted through to an interior surface of the tumor. While only a single probe is shown, it should be recognized that the use of a plurality of probes to induce apoptotic cell death in a relatively larger portion of the tumor can provide superior results, by significantly reducing tumor load and enabling the immune response to be more effective.

In FIG. 1, a syringe 16 is illustrated; the syringe includes a needle 14 that is inserted into tumor 10 to infuse a photoreactive agent, such as a porphyrin or talaporfin sodium (understanding that such agents are exemplary, rather than limiting), into the treatment site. Alternatively, the porphyrin or other photoreactive agent can be administered intravascularly. The photoreactive agent may be selectively absorbed by the abnormal cells comprising tumor 10 to a much greater extent than by surrounding normal cells. Light emitted by light sources 26 has a characteristic waveband that overlaps at least a portion of an absorption waveband of the photoreactive agent. Note that photoreactive agents often exhibit two primary absorption wavebands, e.g., one in the blue range of the spectrum, and one in the red-IR range of the spectrum. The light source employed needs to only overlap one of those absorption bands to be effective. The activated agent either disrupts the cell membrane of the tumor cells, or constricts the tumor's vascular structure, leading to apoptotic tumor cell death, and the release of immune-stimulating factors. It should be noted that high light fluence rates can produce massive damage to cells, mostly causing necrosis. Low fluence rates result in less cell damage, producing mostly apoptosis and minimizing necrosis. The concepts disclosed herein control light fluence levels to prevent cell necrosis.

Note that a part of contiguous portion 18 is proximate a boundary of tumor 10, enabling the immune stimulating factors to reach the body's lymphatic system. Again, as noted above, more probes can be used to increase the size of contiguous portion 18, to further reduce the tumor load, and increase an amount of immune-stimulating factors released.

Thus, in general, the step of administering the light therapy treatment includes the step of administering a photoreactive agent to the treatment site. The photoreactive agent is selected for one or more characteristic wavebands of light absorption. Light having one or more emission wavebands substantially corresponding to and overlapping at least part of the characteristic waveband of light absorption of the photoreactive agent is applied to the treatment site during each of the plurality of light therapy treatments. The light is absorbed by the photoreactive agent, which then destroys the abnormal tissue via apoptotic cell death. Light can be administered from a light source implanted within the abnormal tissue, or disposed adjacent to the abnormal tissue.

Although not shown, instead of using an implanted light source, an optical fiber can be used to administer light to a treatment site (e.g., tumor 10) within the patient's body from an external light source such as a laser. Other types of light sources can be used either in connection with implanted probes like those shown in FIG. 1, or to provide light from outside the patient's body. The only significant requirement is that the light source produces light of sufficient quality or intensity to excite the photoreactive agent within the tumor and/or tumor vasculature.

If an implanted probe is employed, electrical power can be supplied to energize the probe from outside the patient's body using an external power source that is connected to a coil applied on the outer surface of the patient's skin, generally opposite an internally implanted coil that is connected to the implanted probe (neither shown). A similar arrangement can be used to provide power and other signals to implanted probe 20, in FIG. 1. Other details related to the use of implanted probes and other designs for light probes are disclosed in the patents and patent applications identified above (see paragraph 0002).

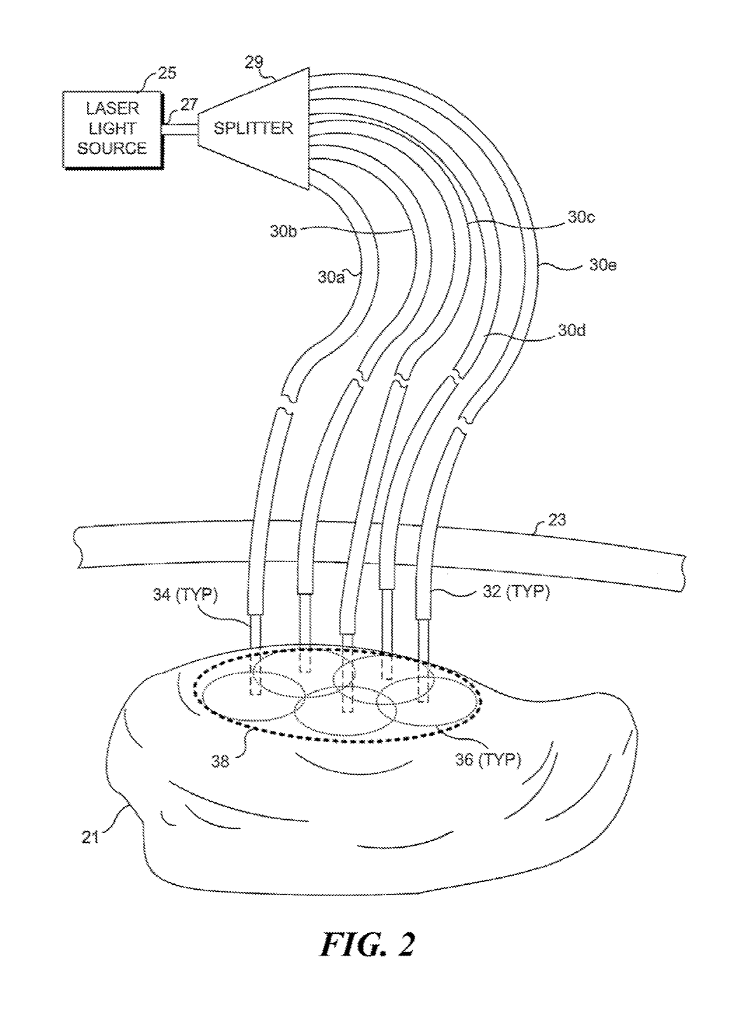

FIG. 2 schematically illustrates the apoptotic treatment disclosed herein being implemented using a plurality of light probes, where the light probes include optical fibers coupled to an external light source (note that the plurality of light probes can also comprise the light probe shown in FIG. 1, which includes a light source rather than an optical fiber coupled to an external light source). Referring to FIG. 2, a tumor 21 is disposed within a patient's body. Tumor 21 is relatively large, having a length of approximately 7 to 10 cm and a transverse width of about 7 cm in this exemplary illustration. The tumor is disposed below a dermal layer 23, for example, within the patient's abdominal cavity.

A photoreactive agent is administered such that the photoreactive agent is present in the abnormal tissue of tumor 21 and/or in its vasculature. Thereafter, using a surgical procedure to access tumor 21 through dermal layer 23, or using an endoscopic procedure with minimally invasive impact, a plurality of optical fibers 30a-30e are inserted into the interior of tumor 21 in a spaced-apart array so that the optical fibers are arranged in a pattern that is more likely to increase the effectiveness of the therapy administered to the tumor. A laser light source 25 produces light absorbed by the photoreactive agent that has been administered to the patient.

Light emitted by laser light source 25 is conveyed through an optical fiber 27 to a splitter 29 that divides light among optical fibers 30a-30e. The light is conveyed through these optical fibers toward their distal ends. Optical fibers 30a-30e include an outer cladding 32 that minimizes losses through the outer surface of the optical fiber, insuring that substantially all of the light input to the optical fibers at their proximal ends, i.e., at splitter 29, is conveyed through the optical fibers to their distal ends, which have been inserted interstitially into the interior of tumor 21.

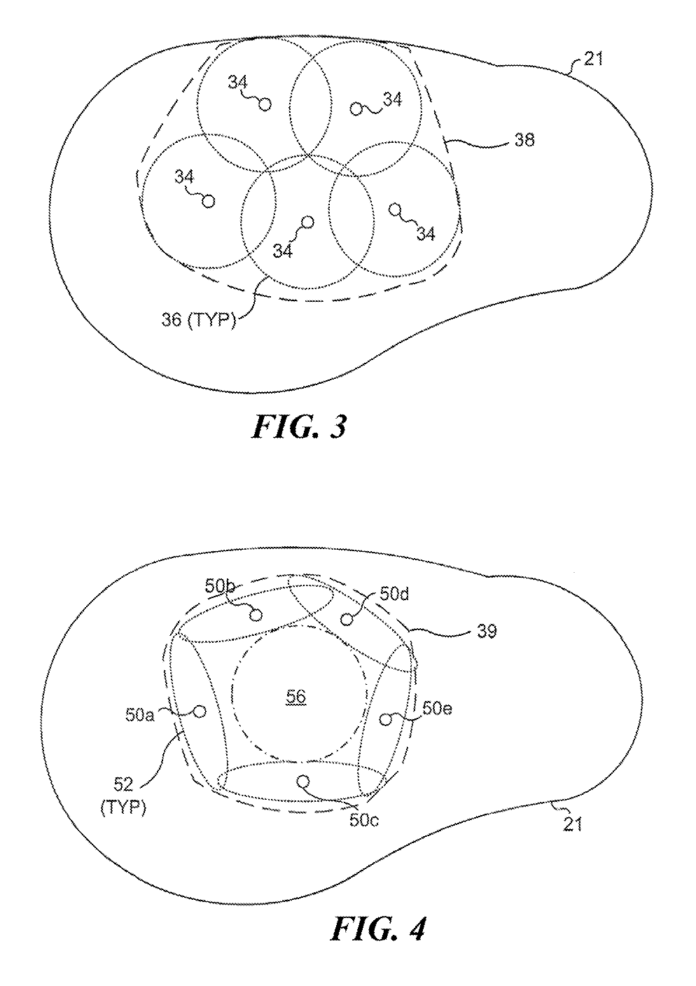

In the embodiment illustrated in FIG. 2, cladding 32 is removed from approximately the last 3 to 4 cm of the distal ends of each of optical fibers 30a-30e, exposing a core 34. A diffusing surface is provided on the exposed portion of core 34, e.g., by roughening the surface of the exposed core, thereby insuring that light conveyed through the optical fibers is uniformly distributed through the sides and through the distal ends of the optical fibers inserted into the tumor. Light emitted by the exposed distal ends of each of these optical fibers penetrates tumor 21 to an effective depth of less than 1.5 cm. The penetration depth of the emitted light into the tumor determines a generally cylindrical expected fluence zone 36, the radius of which is indicated by the dotted circles shown in FIG. 2, and more clearly, in the plan view of FIG. 3.

As will be evident from FIG. 3, the exposed portions of cores 34 from which the cladding has been removed are inserted into tumor 21, generally forming a circle in which the expected fluence zones 36 around each optical fiber at least partially overlap. It should also be noted that the expected fluence zone for each optical fiber is determined partly by the intensity of the light delivered to the distal ends of each of the optical fibers and partly by the nature of the tissue in tumor 21. Measurements in the prior art indicate that for most tumor tissue, the maximum effective depth of light penetration (at a wavelength of 600-700 nm) within tumor tissue is less than 1.5 cm. Furthermore, the effective depth of the expected fluence zones is substantially less than the maximum, depending upon a number of factors such as the blood concentration in the tissue, color of the tissue, the photoreactive agent concentration, etc.

Note that the fluence zone of each optical fiber overlaps at least one other fluence zone of an adjacent optical fiber, to achieve a contiguous portion 38 of treated abnormal tissue. As discussed above, the treatment results in apoptotic cell death in contiguous portion 38. Also note that at least part of contiguous portion 38 is disposed proximate a boundary of tumor 21, enabling the immune-stimulating factors released from the apoptotic cells to reach the body's lymphatic system. The concentrated immune-stimulating factors overcome the tumor's immunosuppressive microenvironment, and eventually make their way to the lymphatic system, to stimulate the immune system.

Referring to FIG. 2, note that if each core 34 were longer, and extended deeper into tumor 21, the fluence zone for each probe would be generally cylindrical in shape, as opposed to circular. Such a probe design (i.e., an elongate probe that can extend relatively deep into a tumor mass) can be used to increase the size of the contiguous portion, thereby reducing tumor load and generating additional quantities of immune-stimulating factors.

It should be noted that the concepts disclosed herein are clearly not limited to administering light using a laser source. Instead, almost any source of light can be used that emits light in the appropriate waveband, i.e., corresponding to or overlapping at least a portion of the absorption waveband of the photoreactive agent. For example, the light source may comprise an electroluminescent device, an LED, a fluorescent light source, an incandescent light source, an arc lamp, or other source of light that is conveyed to a tumor through an optical fiber (or light pipe), or is disposed on a probe that is inserted into the tumor.

FIG. 4 illustrates implanted probes 50a-50d, which have been inserted into tumor 21 such that there is no overlap between fluence zones 52 of each probe (each of probes 50a-50d having a generally circular expected fluence zone 52). While apoptotic cell death can occur in each fluence zone 52 (if the fluence level is controlled to prevent necrosis due to a lethal increase in tissue temperatures), a single contiguous zone of apoptotic tumor cells is not achieved. Thus, the probe configuration shown in FIG. 4 is not as desirable as the configuration shown in FIG. 3, because the apoptotic cells are not concentrated in a single contiguous portion of the tumor, but rather are spread across the mass of the tumor. This spread of the apoptotic cells means that the immune stimulating factors released by the apoptotic cells are not concentrated in any area of the tumor, and it is unlikely that such immune stimulating factors will overpower the immunosuppressive tumor microenvironment. Relatively small amounts of immune-stimulating factors having difficulty overcoming the tumor's immunosuppressive microenvironment and reaching the periphery of the tumor, such that relatively few immune stimulating factors actually make their way to the lymphatic system to stimulate the immune system.

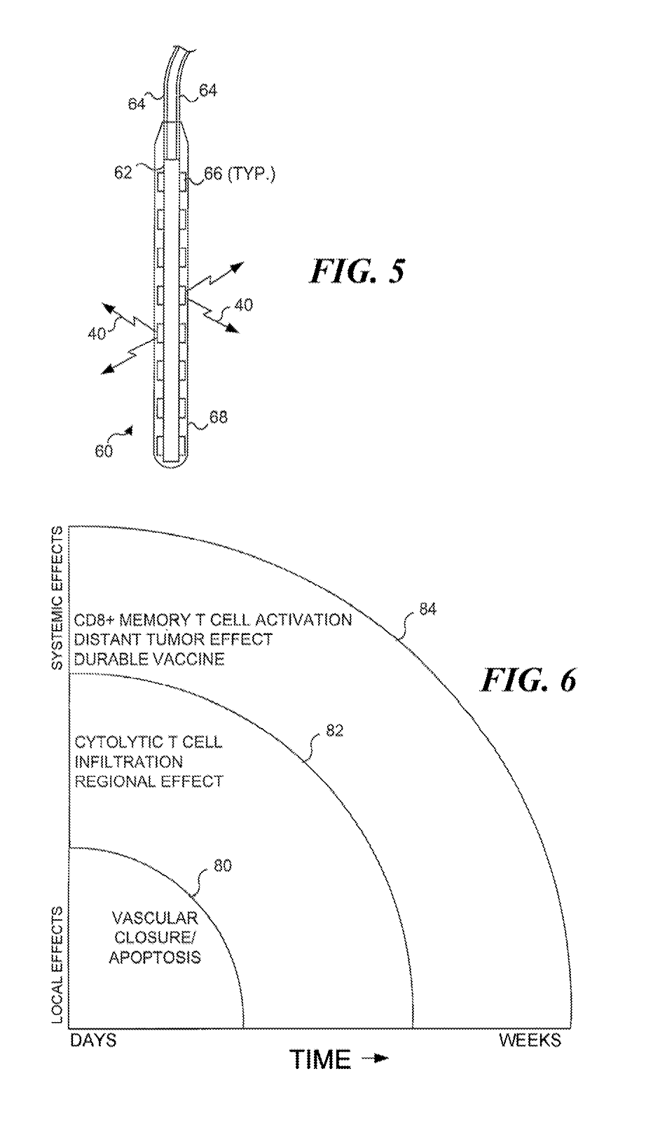

In FIG. 5, details of a probe 60 suitable for use in delivering the light therapy disclosed herein are illustrated. Probe 60 includes a flexible substrate 62 on which are mounted a plurality of spaced-apart LEDs 66. Leads 64 are coupled to conductive traces (not shown) on flexible substrate 62 and provide electrical current to energize LEDs 66, causing them to emit a light 40 of the appropriate waveband that overlaps at least a portion of the light absorption waveband of the photoreactive agent. An optically transparent, biocompatible envelope 68 surrounds LEDs 66 and flexible substrate 62, sealing the structure so that the internal components are not exposed to bodily fluids. It should be recognized that such a probe configuration is exemplary, rather than limiting. For example, other light probe designs useful in practicing the present approach are disclosed in the patents and patent applications identified above in paragraph 0002.

FIG. 6 graphically illustrates a timeline for the treatment disclosed herein. Initially, as indicated by an area 80, the light activated therapeutic agent will kill tumor cells apoptotically, either by damaging the cell membranes to induce apoptosis, or by damaging the vasculature to cut off nutrients to the tumor cells, or both. Next, as indicated by area 82, the immune-stimulating factors released by the dead tumor cells stimulate the production of T cells that attack the balance of the tumor. Note that unless the tumor load is reduced by generating a sufficiently large contiguous zone of apoptotic cells in the tumor, the tumor load will not be reduced enough to enable the immunogenic effects to outweigh the immunosuppressive effect of the tumor. In general, the contiguous zone of apoptotic cells in the tumor should be as large as practical, preferably in excess of about 20% of the tumor volume, more preferably in excess of about 50% of the tumor volume, and even more preferably in excess of about 75% of the tumor volume. Finally, as indicated by an area 84, the tumor load will have been reduced sufficiently that the immune system has been reset, such that memory T cell activation provides a vaccine-like effect, and tumor cells elsewhere in the body are attacked by the immune system in an ongoing fashion.

It will be apparent that the probes and leads in the above examples may be replaced with optical fibers coupled to one or more internal or external light sources. In addition, it should be apparent that many other configurations of probes or optical fibers can be employed to achieve the concomitant effects resulting from long-term administration of light therapy in accord with the concepts disclosed herein.

By reducing tumor load and stimulating the immune system, the concepts disclosed herein will enable the body's immune system to attack and cause abnormal tissue death at the initial treatment site and at other locations in the body as well. This ongoing reduction in the amount of abnormal tissue in the body will lead to one or more of the following clinical end-points:

An increase in an overall survival rate.

An increase in a median overall survival rate.

An increase in a progression free survival rate.

An increase in a disease free survival rate.

A positive post treatment tumor response.

A relief of symptoms associated with the abnormal tissue.

A reduction in symptoms associated with the abnormal tissue.

A clinical benefit.

The reduction or elimination of cachexia.

Although the concepts disclosed herein have been described in connection with the preferred form of practicing them and modifications thereto, those of ordinary skill in the art will understand that many other modifications can be made thereto within the scope of the claims that follow. Accordingly, it is not intended that the scope of these concepts in any way be limited by the above description, but instead be determined entirely by reference to the claims that follow.

The invention in which an exclusive right is claimed is defined by the following:

1. A method for destroying abnormal tissue in an abnormal tissue mass in a patient, comprising the steps of:
(a) administering a photoreactive agent having one or more characteristic light absorption wavebands, to the patient, such that a quantity of the photoreactive agent is present in the abnormal tissue mass;
(b) irradiating a contiguous portion of the abnormal tissue mass with light having a characteristic wavelength or waveband that overlaps at least a portion of at least one characteristic absorption waveband of the photoreactive agent, at least a portion of the contiguous portion of the abnormal tissue mass being disposed proximate to an outer boundary of the abnormal tissue mass; and
(c) controlling conditions for irradiating the contiguous portion of the abnormal tissue mass with the light so as to reduce a release of immunosuppressive factors by the abnormal tissue, while stimulating a release of immune-stimulating factors by apoptotic cells in the abnormal tissue mass.

2. The method of claim 1, wherein the step of controlling the conditions for irradiating comprises the step of controlling light fluence while irradiating the contiguous portions of the abnormal tissue mass to be at a level that preferentially causes apoptotic cell death rather than necrotic cell death in the abnormal tissue mass.

3. The method of claim 1, wherein the step of controlling the conditions for irradiating comprises the step of reducing a number of viable cells in the abnormal tissue mass while causing minimal necrotic cell death of the abnormal tissue.

4. The method of claim 1, further comprising the step of stimulating an immunogenic response by an immune system of the patient, with the release of the immune-stimulating factors by the apoptotic cells in the abnormal tissue mass, enabling the immune system to attack remaining abnormal tissue both in the abnormal tissue mass and elsewhere in the patient.

5. The method of claim 4, wherein the step of stimulating the immunogenic response comprises the step of achieving at least one clinical endpoint selected from a group of clinical endpoints consisting of:
(a) increasing an overall survival rate of the patient;
(b) increasing a medial overall survival rate of the patient;
(c) increasing a progression free survival rate of the patient;
(d) increasing a disease free survival rate of the patient;
(e) generating a positive post treatment tumor response in the patient;
(f) providing relief of symptoms associated with the abnormal tissue mass;
(g) reducing symptoms in the patient that are associated with the abnormal tissue mass;
(h) providing a clinical benefit to the patient; and
(i) reducing a degree of cachexia in the patient.

6. The method of claim 1, further comprising the step of introducing a plurality of light probes into the abnormal tissue mass to emit light used for irradiating the contiguous portion of the abnormal tissue mass.

7. The method of claim 6, wherein the step of introducing the plurality of light probes comprises the step of positioning the plurality of light probes so that they are generally adjacent to each other.

8. The method of claim 6, wherein the step of introducing the plurality of light probes into the abnormal tissue mass comprises the step of overlapping fluence zones of at least some of the plurality of light probes, enabling irradiation of the contiguous portion of the abnormal tissue mass with the light emitted by the plurality of light probes.

9. The method of claim 1, wherein the step of irradiating the contiguous portion of the abnormal tissue mass with light comprises the step of irradiating a continuous portion of the abnormal tissue mass that corresponds to about 50% to about 99% of the abnormal tissue mass.

10. The method of claim 1, wherein the step of irradiating the contiguous portion of the abnormal tissue mass with light comprises the step of irradiating a continuous portion of the abnormal tissue mass that corresponds to about 75% to about 99% of the abnormal tissue mass.

11. The method of claim 1, wherein the step of irradiating the contiguous portion of the abnormal tissue mass with light comprises the step of irradiating a continuous portion of the abnormal tissue mass that corresponds to at least 20% of the abnormal tissue mass.

12. A method for enhancing results of using light activated drug therapy when treating an abnormal tissue mass in a patient, comprising the steps of:
(a) administering a light activatable reagent to the patient, such that a quantity of the light activatable reagent is present in the abnormal tissue mass;
(b) introducing a plurality of probes into the abnormal tissue mass, wherein the plurality of probes emit light having a characteristic waveband for activating the light activatable reagent;
(c) positioning the plurality probes to irradiate a contiguous portion of the abnormal tissue with the light and so that at least a portion of the contiguous portion of the abnormal tissue is disposed proximate an outer boundary of the abnormal tissue mass; and
(d) controlling an intensity of the light emitted by the plurality of probes to activate the light activatable reagent in the contiguous portion of the abnormal tissue mass, the intensity of the light being sufficient for inducing apoptotic cell death in the abnormal tissue mass, while minimizing necrotic cell death in the abnormal tissue mass.

13. The method of claim 12, wherein the step of inducing apoptotic cell death comprises the step of stimulating an immune system of the patient by causing a release of immune-stimulating factors from apoptotic cells in the abnormal tissue mass across the outer boundary of the abnormal tissue mass, causing the immune system of the patient to attack remaining abnormal tissue, both in the abnormal tissue mass and elsewhere in the patient.

14. The method of claim 12, further comprising the step of reducing an amount of immunosuppressive factors associated with the abnormal tissue mass, by reducing an amount of viable cells in the abnormal tissue mass as a result of activating the light activatable reagent.

15. The method of claim 12, wherein the step of minimizing necrotic cell death results in minimizing an amount of tumor-promoting factors associated with necrotic cell death present in the patient, which helps enhance the results of using the light activated drug therapy.

16. The method of claim 12, wherein the light emitted by the plurality of light probes to activate the light activatable reagent in the contiguous portion of the abnormal tissue mass activates the light activatable reagent in a continuous portion of the abnormal tissue mass that corresponds to about 50% to about 99% of the abnormal tissue mass.

17. The method of claim 12, wherein activation of the light activatable reagent by the light emitted from the plurality of probes causes an immunogenic response that enhances the light activated therapy by achieving at least one clinical endpoint selected from a group of clinical endpoints consisting of:
(a) increasing an overall survival rate of the patient;
(b) increasing a medial overall survival rate of the patient;
(c) increasing a progression free survival rate of the patient;
(d) increasing a disease free survival rate of the patient;
(e) generating a positive post treatment tumor response in the patient;
(f) providing relief of symptoms associated with the abnormal tissue mass;
(g) reducing symptoms in the patient that are associated with the abnormal tissue mass;
(h) providing a clinical benefit to the patient; and
(i) reducing a degree of cachexia in the patient.

18. A method for using a light activated drug therapy to treat abnormal tissue mass within a patient, so as to stimulate a more effective immunogenic response by the patient's body, comprising the steps of:
(a) administering a light activatable reagent to the patient, such that a quantity of the light activatable reagent is present in the abnormal tissue mass, the light activatable reagent having one or more characteristic wavebands of light absorption;
(b) irradiating a contiguous portion of the abnormal tissue mass with light having one or more characteristic wavebands that overlap at least one of the characteristic wavebands of light absorption of the light activatable reagent, the contiguous portion comprising at least about 50% of the abnormal tissue mass and at least a portion of the contiguous portion of the abnormal tissue mass being disposed proximate to an outer boundary of the abnormal tissue mass; and
(c) controlling the irradiation of the contiguous portion of the abnormal tissue mass that activates the light activatable reagent, so as to induce an apoptotic cell death of the abnormal tissue in the abnormal tissue mass and thereby stimulating the immune system of the patient's body, causing an immunogenic response that attacks the abnormal tissue.

19. The method of claim 18, wherein the step of controlling the irradiation so as to induce apoptotic cell death of the abnormal tissues reduces an amount of viable cells in the abnormal tissue mass, reducing an amount of immunosuppressive factors associated with the abnormal tissue mass present in the patient's body.

20. The method of claim 18, wherein the step of controlling the irradiation includes the step of minimizing necrotic cell death in the abnormal tissue mass, which minimizes an amount of tumor-promoting factors associated with necrotic cell death in the patient's body.

21. The method of claim 18, further comprising the step of introducing a plurality of probes that emit light into the abnormal tissue mass, before the step of irradiating the contiguous portion of the abnormal tissue mass with the light.

22. The method of claim 21, further comprising the step of disposing the plurality of light probes so that at least some of the plurality of probes are adjacent to each other.

23. The method of claim 18, wherein the step of controlling the irradiation to cause the immunogenic response achieves at least one clinical endpoint selected from a group of clinical endpoints consisting of:
(a) increasing an overall survival rate of the patient;
(b) increasing a medial overall survival rate of the patient;
(c) increasing a progression free survival rate of the patient;
(d) increasing a disease free survival rate of the patient;
(e) generating a positive post treatment tumor response in the patient;
(f) providing relief of symptoms associated with the abnormal tissue mass;
(g) reducing symptoms in the patient that are associated with the abnormal tissue mass;
(h) providing a clinical benefit to the patient; and
(i) reducing a degree of cachexia in the patient.
